(19) United States
(12) Patent Application Publication
(10) Pub. No.: US 2015/0241682 A1
(43) Pub. Date: Aug. 27, 2015

(54) IMMERSION MEDIUM AND ITS LAYOUT IN AN OPTICAL SYSTEM

(71) Applicant: Carl Zeiss Microscopy GmbH, Jena (DE)

(72) Inventors: Thorsten Kues, Bovenden-Eddigehausen (DE); Robin zur Nieden, Gottingen (DE)

(21) Appl. No.: 14/634,378

(22) Filed: Feb. 27, 2015

(30) Foreign Application Priority Data
Feb. 27, 2014 (DE) 102014002744.9

(51) Int. Cl. G02B 21/02; C08L 83/04; C07C 9/00; C08K 5/41; C08F 110/02; C08G 77/04; C08F 120/10

(52) U.S. Cl. CPC G02B 21/02; C08G 77/04; C08L 83/04; C08F 120/10; C08K 5/41; C08F 110/02; C07C 9/00

(57) ABSTRACT

An immersion medium for microscopic or macroscopic examination of an object having an index of refraction between 1.0 to 1.70, a transmission between Lambda=0.30 to 1.2 μm, a transmission T_{Total}=0.8 and higher, a temperature range from 0 degrees to 50 degrees Celsius, resistance to acids/bases and heat, a shear modulus of 129 to 500 Kpa, resistance to chemicals and environmental friendliness, as well as low inherent fluorescence. The immersion medium may be configured as an elastomer immersion. Embodiments of invention can include the layout of the immersion medium in the working position of an optical system.

### RELATED APPLICATIONS

This present application claims priority to German Application No. 10 2014 002 744.9, filed Feb. 27, 2014, said priority application being fully hereby incorporated herein by reference in its entirety.

### FIELD OF THE INVENTION

The invention relates to an immersion medium for microscopic or macroscopic examination of an object, Furthermore, the invention relates to the layout of the immersion medium in a working position in an optical system.

### BACKGROUND OF THE INVENTION

In microscopy, the use of immersion objectives has numerous advantages for the experimental data that can be achieved. Important examples of the fundamental advantages of immersion are:
The apertures that can be achieved are higher, leading to:
   Higher spatial resolution
   Greater light collection efficiency
      High signal/noise ratio or signal/background ratio
      Short exposure times
      Great temporal resolution
      Reduced phototoxicity
   Reduction of image defects, as for example due to spherical aberration caused by differences in the index of refraction in the beam path, particularly in the case of great penetration depths, and
   Chromatic aberration, particularly axial chromatic aberration.
Reduction of reflections/scattered light at boundary layers:
   Differences in the index of refraction at boundary layers generally cause disruptive reflections, and
   Immersion media reduce the reflections and thereby improve the signal/background ratio and the contrast.

In DE 10343722 A1, for example, a solid-body immersion lens for a microscope having an objective system having a predetermined numerical aperture is described for this purpose, wherein the index of refraction of the material of the solid-body immersion lens is selected in such a manner that the numerical aperture is increased when the solid-body immersion lens is placed ahead of the objective system.

Typical immersion media are water, organic substitute media for water, glycerin, and special immersion oils. DE 102011113116 B3, for example, describes an immersion body that consists of a box-shaped housing that has a stable wall and two transparent cover surfaces, wherein the first transparent cover surface consists of an elastic material, and the housing is filled with an immersion fluid.

However, aside from the stated advantages for the data quality that can be achieved, numerous disadvantages also result from the use of these immersion media. These disadvantages frequently outweigh the stated advantages in practice. In practice, practical use of immersion objectives is therefore greatly restricted. Typical problems with conventional immersion media are:
Disadvantages of immersion oil
   Contamination of the objective and the sample,
   Relatively complicated cleaning of the samples and the objective, and
   Automatic immersion can be implemented only in very complicated manner.
Disadvantages of water and of all liquids having a high vapor pressure (index of refraction n=1.33)
   Relatively high vapor pressure, i.e. strong evaporation, therefore
      unsuitable for long-term experiments, and
      complicated auto-immersion systems are necessary.
   Electrical conductivity
Disadvantage of water substitute materials (index of refraction n=1.33)
   Viscosity is not temperature-stable, such as, for example, Immersol W.
Disadvantages of glycerin (n=1.456)
   Hydroscopic
      Mechanical properties, such as viscosity and friction, for example, change.
      Optical properties, such as index of refraction, dispersion, and absorption, for example, change.

All immersion media are generally liquid at the conventional temperature. This results in the following problems, among others:
Experimental/applicative restrictions such as:
   Larger working distances, which are required for electro-physiology, stereo-microscopy, and macroscopy, for example, cannot be implemented,
   Multi-position experiments are limited, because the immersion medium generally remains at the first contact location,
   Depending on the medium, long-term experiments are limited, because the medium changes over time,
   Use in automation can only be implemented with great effort, and
   All stated immersion media can be used exclusively in a narrow temperature band.
Technical risks when using liquids on/in the microscopes are:
   Damage to objective and equipment due to penetrating liquid,
   Effort for risk minimization is great (“immersion stop”)
      Costs
      Design restrictions, and
   Due to great viscosity, loosening of the cover glass can occur.
Use of the immersion media fundamentally deters the user due to:
   more difficult, complicated handling, particularly for inexperienced users,
   it costs time,
   it restricts experimental possibilities,
   cleaning of the sample and or the equipment can be time-consuming, depending on the medium, and
   access to the sample space is frequently severely restricted because of incubation, laser protection, etc.

Proceeding from this, the invention is based on the task of finding an immersion medium for microscopic or macroscopic examination of an object, which avoids the disadvantages of the known solutions while maintaining the advantages of immersions. Furthermore, the task consists in making available a layout of the immersion medium in an optical system.

### SUMMARY OF THE INVENTION

According to embodiments of the invention, the immersion medium is an elastomer immersion, consisting of an elastomer, advantageously a non-toxic elastomer, which, in an advantageous embodiment, is a shape-stable, elastically deformable plastic in the form of a siloxane and/or a natural polymer, the glass transition point of which is situated below the temperature of use.

An immersion medium for microscopic or macroscopic examination according to embodiments of the invention has an index of refraction between 1.0 to 1.70, a transmission between Lambda=0.30 to 1.2 μm, a transmission T_{Total}=0.8 and higher, a temperature range from 0 degrees to 50 degrees Celsius, resistance to acids/bases and heat, a shear modulus of 129 to 500 Kpa, resistance to chemicals and environmental friendliness, as well as low inherent fluorescence.

Because of the physical-chemical properties of the elastomer immersion, numerous layouts are possible, which particularly allow combining different elastomer immersions, for example having different indices of refraction and/or viscosities. Numerous elastomers furthermore have excellent casting and molding properties. This actually allows a nano-structured/micro-structured elastomer immersion.

In advantageous uses, a polydimethylsiloxane (PDMS) is used as the siloxane. Furthermore, elastomers for immersion consist of mineral oil products, such as polymethylmethacrylate (PMMA), polyethylene gel or paraffin gel, in an advantageous use. Furthermore, the natural polymers can consist of gelatin, agarose or vegetable polysaccharides (pectins), in advantageous uses.

According to embodiments of the invention, the elastomer immersion is either a fixed or an interchangeable component of the object vessel, of the object, or of the optical system. Both the working position and the composition of the elastomer immersion can be configured to be very variable on the basis of the physical-chemical properties. In the simplest case, a homogeneous elastomer immersion can be used analogously to the liquid immersion, such as, for example:
1. between an objective and an object/object vessel, in direct contact, in each instance;
2. between a condenser and the object/object vessel, in direct contact, in each instance; or
3. in a combination of 1 and 2.

Furthermore, combinations of different elastomers, i.e. of different elastomer properties, to produce what are called heterogeneous elastomer immersions, are conceivable. This is advantageous, for example, in order to minimize the friction between the object and the microscope components when using highly viscous elastomer immersions.

Furthermore, mechanical properties of the immersion medium also move into the foreground. The elastomer dispersion described can be gel-like (low viscosity) or highly viscous. This can require different mechanical and/or optical adaptations of the mechanical interfaces of the imaging system, depending on the location of use and the selected viscosity, for example adaptations of the object vessel, the objective and/or the condenser.

It could be practical to use a convex front lens on the objective and/or on the condenser to displace the air in the case of a homogeneous highly viscous immersion. Furthermore, a holder for the elastomer cushion on the objective and/or on the condenser is contemplated.

The elastomer immersion according to the invention can be configured in variable manner, so that adaptations to the temperature-dependent index of refraction, the dispersion (Abbe number), the transmission as well as the viscosity of the application are possible. This takes place after selection of the substance class, such as silicones, siloxanes, PU resins, and water-based gels.

In every case, the larger viscosity in comparison with water and the suitability for the imaging part when using a light microscope, such as index of refraction, spectral transmission, and dispersion, can be advantageous. The elastomer immersion according to embodiments of the invention can be used both in the imaging beam path and in the illumination-side beam path.

### BRIEF DESCRIPTION OF THE DRAWINGS

In the following, the layout of the elastomer immersion according to the invention in an imaging system will be explained in greater detail, using exemplary embodiments. The drawings show:

FIG. 1 is a schematic representation of the elastomer immersion between an objective and an object/object vessel;

FIG. 2 is a schematic representation of the elastomer immersion between a condenser and the object/object vessel;

FIG. 3 is a schematic representation of a combination of the layouts according to FIG. 1 and FIG. 2;

FIG. 4 is a schematic representation of the layout of the elastomer immersion according to FIG. 1 with different immersion media;

FIG. 5 is a schematic representation of the layout of the elastomer immersion according to FIG. 2 with different immersion media;

FIG. 6 is a schematic representation of the layout of the elastomer immersion according to FIG. 3 with different immersion media;

FIG. 7 is a schematic representation of the layout of the elastomer immersion on the object/object vessel, with an air layer between the objective and the elastomer immersion;

FIG. 8 is a schematic representation of the layout of the elastomer immersion on the object/object vessel, with an air layer between the condenser and the elastomer immersion;

FIG. 9 is a schematic representation of the layout of the elastomer immersion on both sides of the object/object vessel;

FIG. 10 is a schematic representation of the layout of the elastomer immersion between the objective and the object/object vessel, with air layers;

FIG. 11 is a schematic representation of the layout of the elastomer immersion between the condenser and the object/object vessel, with air layers;

FIG. 12 is a schematic representation of the layout of the elastomer immersion between the condenser and the object/object vessel and between the objective and the object/object vessel, with air layers;

FIG. 13 is a schematic representation of an elastomer immersion provided with an air layer, between the object/object vessel and the objective;

FIG. 14 is a schematic representation of an elastomer immersion provided with an air layer, between the object/object vessel and the condenser;

FIG. 15 is a schematic representation of two elastomer immersions, each provided with an air layer, between the object/object vessel and the objective and between the object/object vessel and the condenser;

FIG. 16 is a schematic representation of the layout of two elastomer immersions between the objective and the object/object vessel;

FIG. 17 is a schematic representation of the layout of two elastomer immersions between the condenser and the object/object vessel;

FIG. 18 is a schematic representation of the layout of two elastomer immersions between the condenser and the object/object vessel and between the objective and the object/object vessel;

FIG. 19 is a schematic representation of the layout of two elastomer immersions between the objective and the object/object vessel, with an embedded fluid chamber;

FIG. 20 is a schematic representation of the layout of two elastomer immersions between the condenser and the object/object vessel, with an embedded fluid chamber;

FIG. 21 is a schematic representation of the layout of two elastomer immersions between the condenser and the object/object vessel and between the objective and the object/object vessel, with fluid chambers embedded on both sides; and

FIG. 22 includes different representations regarding attachment of the elastomer immersion.

### DETAILED DESCRIPTION OF THE DRAWINGS

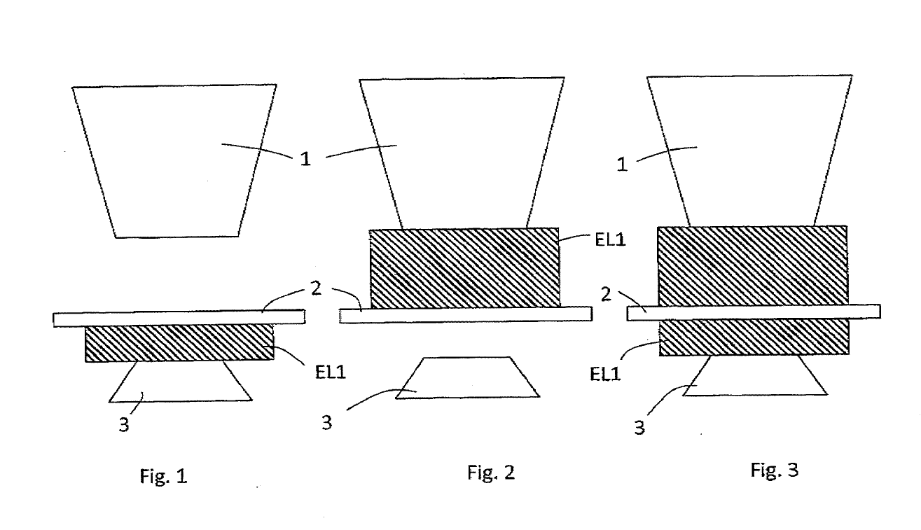

FIG. 1 shows the layout of a condenser 1, an object/object vessel 2, and an objective 3, wherein an elastomer immersion layer E1 is situated in direct contact with the object/object vessel 2 and the objective 3. In FIG. 2, the elastomer immersion layer E1 is situated between the object/object vessel 2 and the condenser 1, in direct contact. In FIG. 3, a combination of the layout according to FIG. 1 and the arrangement according to FIG. 2 is depicted, with the elastomer immersion layer E1 between the condenser 1 and the objective 2.

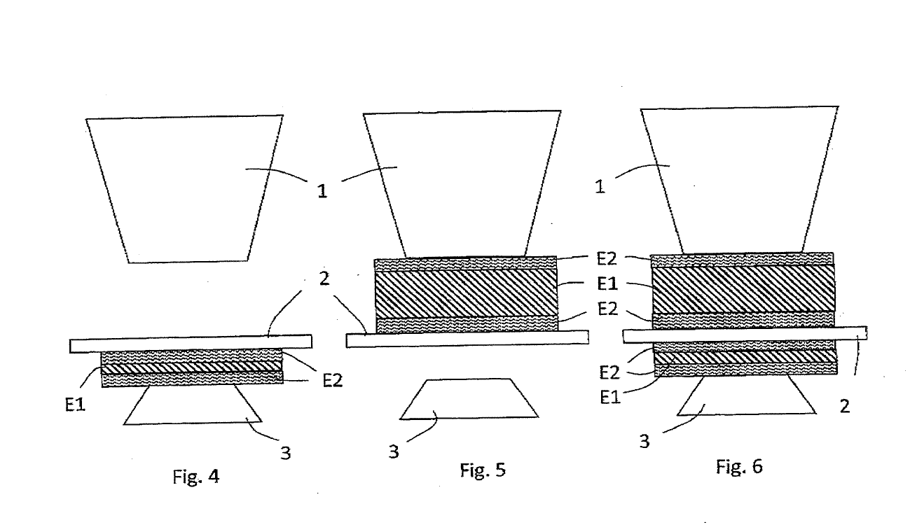

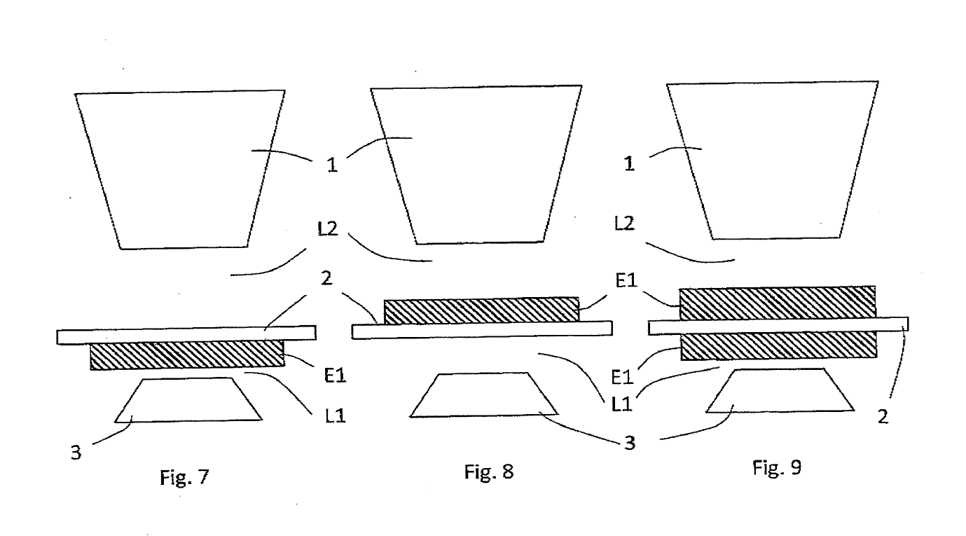

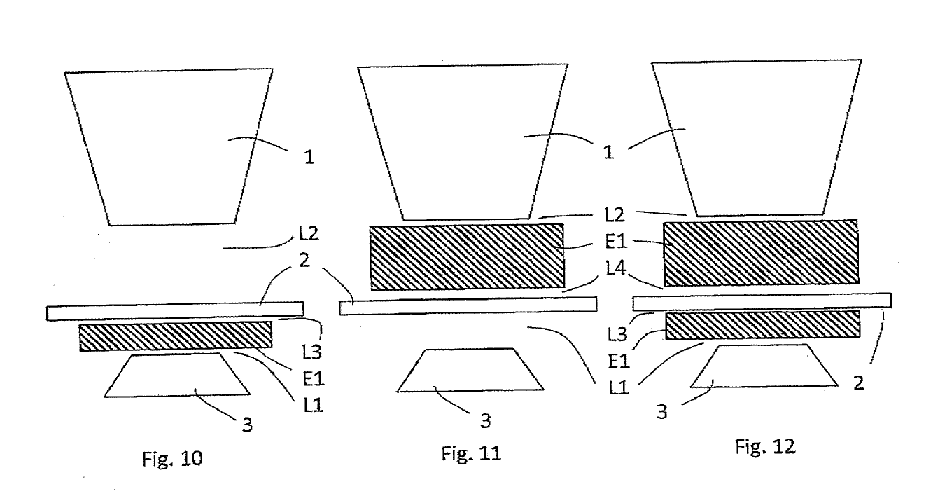

FIGS. 4, 5, and 6 show layouts of the elastomer immersion according to FIGS. 1, 2, and 3 with two different immersion media layers E1 and E2. In FIGS. 7, 8, and 9, layouts of the elastomer immersion layers E1 according to FIGS. 1, 2, and 3 can be seen, in which air layers L1 and L2 are present between the objective 3 and the object/object vessel 2, or between the condenser 1 and the object/object vessel 2, respectively. In this regard, the elastomer immersion layers E1 have direct contact with the object/object vessel 2.

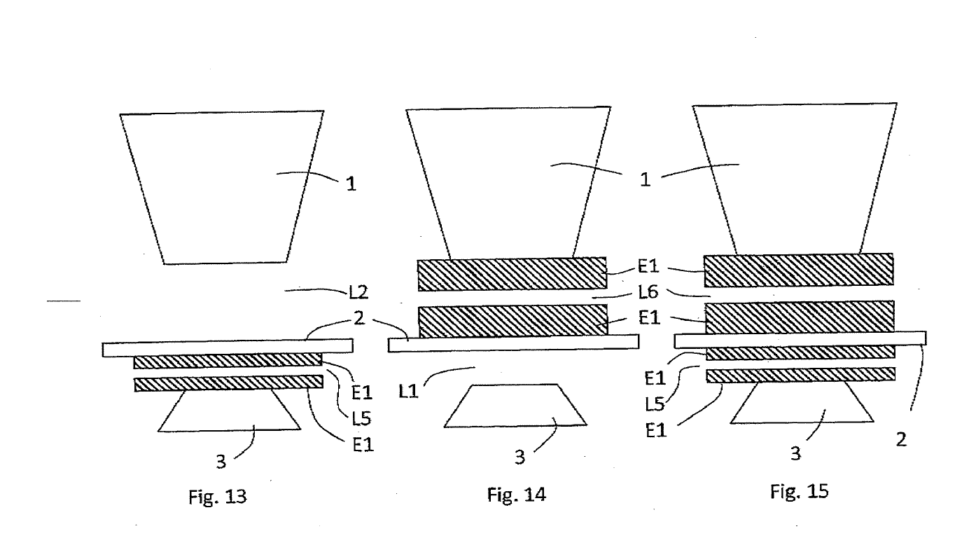

FIGS. 10, 11, and 12 show layouts of the elastomer immersion layers E1 according to FIGS. 7, 8, and 9, in which air layers L3 and L4 are additionally present between the object/object vessel 2 and the elastomer immersion layer E1 and between the object/object vessel 2 and the elastomer immersion layer E1.

The air layers L3 and L4 allow better contacting, because the critical boundary surfaces between glass and elastomer immersion as well as between elastomer immersion and object/object vessel 2 remain constant. The interfaces are then formed between the elastomer immersions.

In FIGS. 13, 14, and 15, layouts of the elastomer immersion layers E1 with additional air layers L5 and L6 in the elastomer immersion layers E1 themselves, according to FIGS. 7, 8, and 9, can be seen.

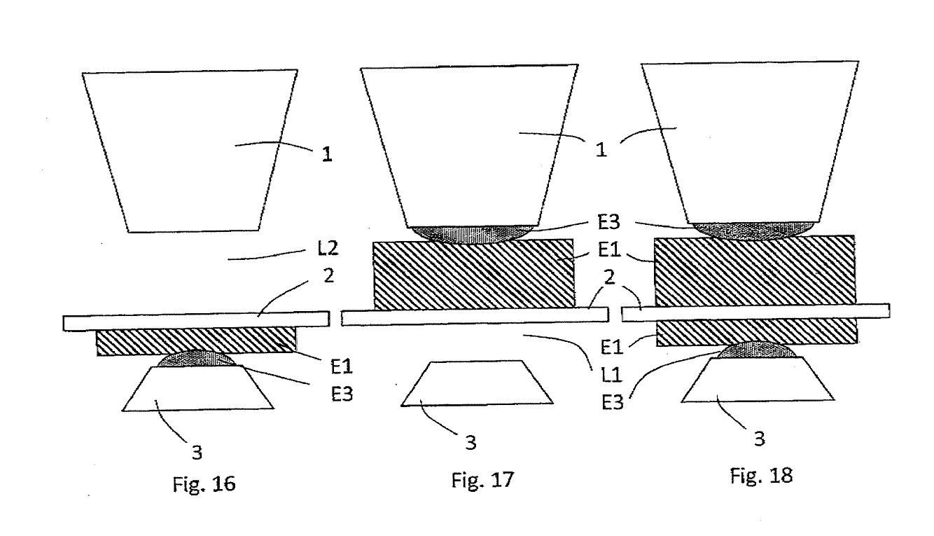

FIGS. 16, 17, and 18 show layouts of two elastomer immersions E1 and E3 that are connected with one another, analogous to the layouts according to FIGS. 4, 5, and 6, in direct contact with the object/object vessel 2, respectively with the condenser 1 and the objective 3, wherein the connections between the elastomer immersion layers E1 and E3 are structured in circular shape.

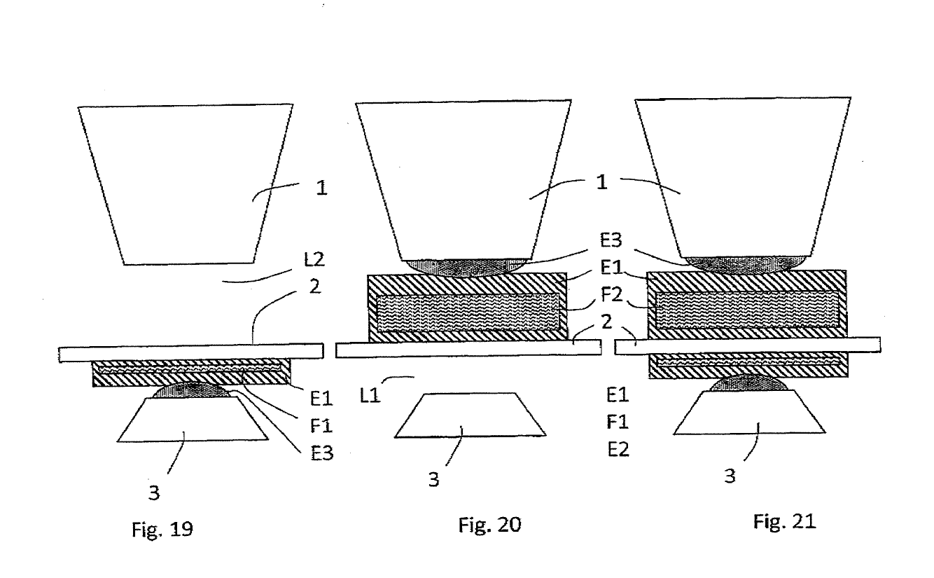

In FIGS. 19, 20, and 21, layouts of two elastomer immersions E1 and E3 that are connected with one another can be seen, analogous to FIGS. 16, 17, and 18, wherein the elastomer immersion E1 is provided with fluid chambers F1 and F2 for accommodating different liquids, in order to optimize the optical properties.

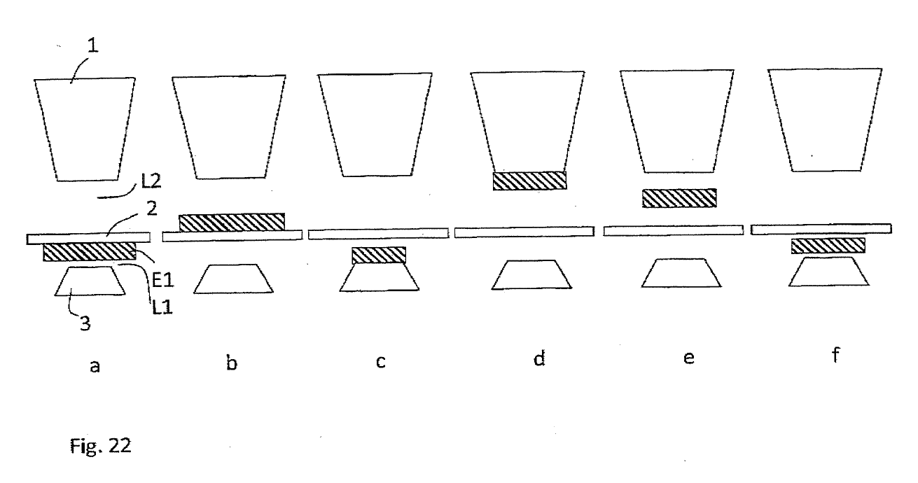

FIG. 22, in alternatives a to f, shows different possibilities for attaching the elastomer immersion E1 in the imaging system, wherein combinations with one another are also conceivable.

What is claimed is:

1. An immersion medium for microscopic or macroscopic examination of an object, having an index of refraction between 1.0 to 1.70, a transmission between Lambda=0.30 to 1.2 μm, a transmission T_{Total}=0.8 and higher, a temperature range from 0 degrees to 50 degrees Celsius, resistance to acids/bases and heat, a shear modulus of 129 to 500 Kpa, resistance to chemicals and environmental friendliness, as well as low inherent fluorescence, wherein the immersion medium is configured as an elastomer immersion.

2. The immersion medium of claim 1, wherein in that the elastomer immersion is a non-toxic elastomer.

3. The immersion medium of claim 1, wherein the elastomer is a shape-stable, elastically deformable plastic in the form of a siloxane or a natural polymer, the glass transition point of which is situated below the temperature of use.

4. The immersion medium of claim 3, wherein the natural polymers are gelatin.

5. The immersion medium of claim 3, wherein the natural polymers are agarose.

6. The immersion medium of claim 3, wherein the polymers are vegetable polysaccharides (pectins).

7. The immersion medium of claim 1, wherein the elastomer immersion is a polydimethylsiloxane (PDMS) with or without an aqueous component.

8. The immersion medium of claim 1, wherein the elastomer immersion is a polymethylmethacrylate (PMMA).

9. The immersion medium of claim 1, wherein the elastomer dispersion is a polyacrylamide gel.

10. The immersion of claim 9, wherein the elastomer immersion is a sodium dodecyl sulfate (SDS).

11. The immersion medium of claim 1, wherein the elastomer immersion is a polyethylene gel, a mineral oil gel or a paraffin gel.

12. The immersion medium of claim 1, wherein the elastomer immersion includes a plurality of elastomers, the elastomer immersion having a heterogeneous structure.

13. The immersion medium of claim 12, wherein portions of the heterogeneous elastomer immersion have air layers.

14. The immersion medium of claim 1, wherein the elastomer immersion has fluid chambers.

15. An optical system including an elastomer immersion, wherein the elastomer immersion is a fixed component of an object or object vessel of the optical system or of a condenser of the optical system.

16. The optical system of claim 15, wherein a plurality of air layers are present between the object or object vessel and the elastomer immersion, or between the elastomer immersion and an objective of the optical system, or between the object or object vessel and the elastomer immersion and a condenser of the optical system.

17. An optical system including an elastomer immersion, wherein the elastomer immersion is an interchangeable component of an object or object vessel of the optical system or of a condenser of the optical system.

18. The optical system of claim 17, wherein in a plurality of air layers are present between the object or object vessel and the elastomer immersion, or between the elastomer immersion and an objective of the optical system, or between the object or object vessel and the elastomer immersion and a condenser of the optical system.
